(19) United States
(12) Patent Application Publication
(10) Pub. No.: US 2024/0115129 A1
(43) Pub. Date: Apr. 11, 2024

(54) FACE IMAGE PROCESSING, EYE FATIGUE DETECTION, STATISTICAL DATA COLLECTION AND METHODS FOR MYOPIA PREVENTION

(71) Applicant: Jieming Mei, Boise, ID (US)

(72) Inventor: Jieming Mei, Boise, ID (US)

(21) Appl. No.: 18/045,466

(22) Filed: Oct. 11, 2022

(51) Int. Cl. A61B 3/113; A61B 3/00; A61B 3/14; G06T 7/00; G06V 40/16

(52) U.S. Cl. CPC A61B 3/113; A61B 3/0016; A61B 3/14; G06T 7/0016; G06V 40/172; G06T 2207/30041

(57) ABSTRACT

Face image processing, eye fatigue detection, statistical data collection and methods for myopia prevention are disclosed, such as face image processing that selects and generates face images of same size, such as eye fatigue detection that calculates the rate of spontaneous eye blink and compares it to a threshold that is based on statistical data and analysis, such as statistical data collection that collects data including but not limited to participants' age, blink rate records, visual acuity test results, and information about general reading or watching environment. The eye fatigue detection applies filter to improve the accuracy of eye blink detection and generates interruption request to stop near work when the blink rates exceeds the threshold. The accuracy of the threshold is guaranteed by the correlation in the statistical data.

### TECHNICAL FIELD

This invention relates to medical technology, and, more particularly, in one or more embodiments, to face image processing, spontaneous blink detection, statistical data collection and methods for eye fatigue detection and eye stress relieving.

### BACKGROUND OF THE INVENTION

Myopia, also known as nearsightedness, is an eye focusing disorder in which light rays from distant objects bent incorrectly, focusing images in front of the retina rather than on the retina and causing blurry vision. It is a common cause of correctable vision loss, projected to affect up to 50% of the world population by 2050 from about 30% in 2020. Worldwide, one-fifth of blindness is predominantly due to myopia (Lancet Glob Health. 2017; 5(9): e888-e897). Annual direct costs of myopia, which includes examinations, vision correction, care for complications such as retinopathy, cataracts and glaucoma, is projected to be $870 billion in 2050 from $359 billion in 2019. Clearly myopia prevention will have a widespread benefit.

It is believed that myopia may be caused by a mix of hereditary and environmental factors. Higher prevalence rate of myopia has been consistently reported in urban than in rural regions (Optom. Vis. Sci. 2015; 92(3):258-66). Evidence is mounting that increased time outdoors reduces the onset of myopia (Ophthalmic Epidemiol. 2013; 20(6):348-59). Environmental factors such as prolonged reading or near work as well as fewer hours spent outdoors are associated with a higher prevalence of myopia. A 2% increased odds of myopia was found for every additional diopter-hour of time spent on near work per week (PLoS One. 2015; 10(10):e0140419). As indicated by additional research, taking regular breaks while doing near work may have a positive effect on myopia. Children who did continuous and focused reading had higher levels of myopia than those children who took frequent breaks, even if they had the same amount of accumulative reading time. Prolonged reading or use of display devices causes eye fatigue. Discrepancy between the perception of eye fatigue and the actual eye fatigue often leads to eye overuse.

Eye fatigue manifests itself in the change of spontaneous blink, a type of blink that occurs regularly without the need for any stimulus, in which both upper eyelids close in a very similar and coordinated manner. A typical prior art includes spontaneous blink detection and blink rate calculation, but there lacks two key components. One missing component is an eye fatigue detection method that is statistically accurate, and the other missing component is an eye strain relieving method that is inexpensive and effective.

There is therefore a need for an improved myopia prevention method with accurate detection of spontaneous blink that provides the basis for eye fatigue detection and relieving method that ensure high statistical accuracy and effectiveness at low cost.

### BRIEF DESCRIPTION OF THE DRAWINGS

FIG. 1 is a block diagram of myopia prevention method in accordance with the invention.

FIG. 2 is a schematic diagram of an image processing method according to an embodiment of the invention.

FIG. 3 is a schematic diagram of a typical prior art facial landmarks.

FIG. 4 is a schematic diagram of a blink detection method according to an embodiment of the invention.

FIG. 5 is an example diagram of spontaneous blink detection result according to an embodiment of the invention.

### DETAILED DESCRIPTION

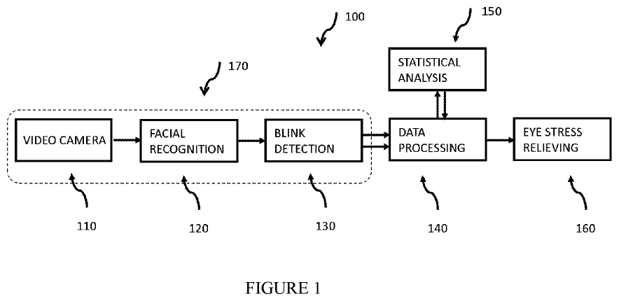

The system diagram 100 of an embodiment of the invention is shown in FIG. 1. The video camera 110 is a camera attached to a monitor, printed material, or the built-in camera in devices such as smart TV, PC, smartphone, or tablet able to generate full-face images. Examples of printed material include but not limited to textbooks, workbooks, reference books, journals and magazines.

On average, each blink lasts between 100 milliseconds and 400 milliseconds. To not miss a blink, the video camera 110 should be able to take more than 10 frame per second (FPS). Preferably the frame rate of the video camera 110 should be 30 FPS or higher. The resolution of the images produced by the video camera 110 can be 640×480 pixels or better.

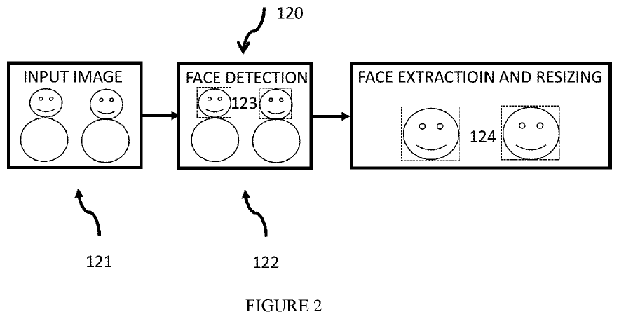

The stream of face images captured by the video camera 110 is sent to the facial recognition function 120 that performs three functions, i.e., face detection, face extraction, and image re-sizing as shown in FIG. 2. The face detection function 122 searches each input image 121 and returns the location and size information of a list of rectangles in which a full face is found. When there are multiple faces in a input image, each face will be detected. The face in each rectangle should enclose necessary landmarks for blink detection. The face extraction function uses the rectangle information to get the portion of each image enclosed by each rectangle 123. Each portion of the image that has a face in it is scaled by the image re-sizing function to reach fixed size of, for example, 300×300 pixels. These three functions can be realized by using prior art such as OpenCV's cascade classifier, cropping and re-sizing methods, respectively. The re-sized images are sent to the blink detection function 130. For each input image to the facial recognition function 120, the number of re-sized images equals the number of faces in the input image.

The blink detection function 130 calculates the coordinates of face organ points for the face in each input image and detects face similarity in them. Depending on the setting, images with faces that are recognized as of a person may be accepted or rejected. In case an image is accepted, the blink detection function 130 calculates the eye aspect ratio (EAR) from the coordinates, applies filter to EAR to detect spontaneous blinks, and outputs blink detection results as time series data to data processing function 140.

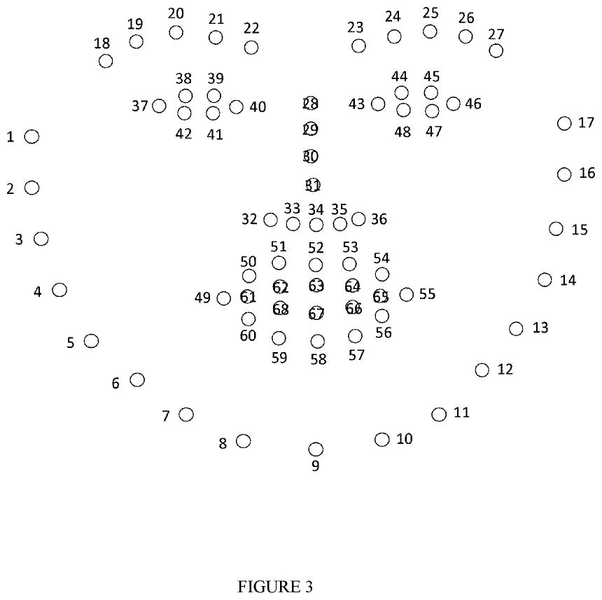

There are multiple trained datasets with annotation of different number of facial landmarks in prior art. Such dataset can be used with histogram of oriented gradients (HOG) features and support vector machines (SVM) to estimate the coordinates of certain number of face organ points. An example of 68 facial points is shown in FIG. 3 wherein 12 points 37-48 mark eye locations with points 37 and 40 on the left eye corners, points 43 and 46 on the right eye corners, points 38 and 39 on the upper eyelid of the left eye, points 44 and 45 on the upper eyelid of the right eye, points 41 and 42 on the lower eyelid of the left eye, and points 47 and 48 on the lower eyelid of the right eye.

EAR can be calculated from the coordinates of the points around the eye. As defined in prior art, EAR for the left eye equals

$$\frac{‖ {P}_{38} - {P}_{42} ‖ + ‖ {P}_{39} - {P}_{41} ‖}{‖ {P}_{37} - {P}_{40} ‖}$$

where ∥P₃₈−P₄₂∥, ∥P₃₉−P₄₁∥, and ∥P₃₇−P₄₀∥ represent the Euclidean distance between points 38, 42, points 39, 41, and points 37, 40, respectively. This value is almost constant with small individual differences when the eye is open. The value approaches zero when the eye is closed. Other advantages of EAR include its independence of head posture or the distance between face and camera. Similarly, EAR for the right eye is defined as

$$\frac{‖ {P}_{44} - {P}_{48} ‖ + ‖ {P}_{45} - {P}_{47} ‖}{‖ {P}_{43} - {P}_{46} ‖}$$

where ∥P₄₄−P₄₈∥, ∥P₄₅−P₄₇∥, and ∥P₄₃−P₄₆∥ are the Euclidean distance between points 44, 48, points 45, 47, and points 43, 46, respectively. Since spontaneous blinks refer to the closing of both upper eyelids, EAR can be calculated as the average, i.e.,

$$EAR = \frac{‖ {P}_{38} - {P}_{42} ‖ + ‖ {P}_{39} - {P}_{41} ‖}{2 ‖ {P}_{37} - {P}_{40} ‖} + \frac{‖ {P}_{44} - {P}_{48} ‖ + ‖ {P}_{45} - {P}_{47} ‖}{2 ‖ {P}_{43} - {P}_{46} ‖} .$$

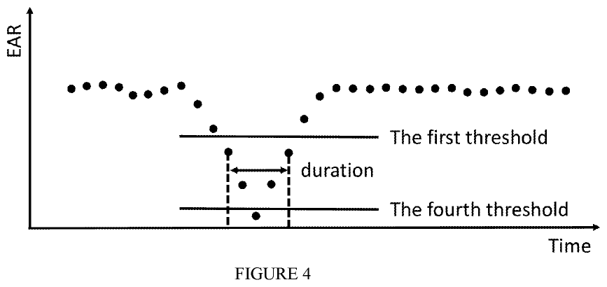

An example of calculated EAR is shown in FIG. 4.

EAR values of accepted images with faces that are recognized as of a person are arranged in one queue where EAR values form a time series. In each queue, the time order of the EAR values match the time order of the input face images from which the EAR values are calculated.

Irregular movements such as looking down, looking away, or stretching cause noise in EAR. This type of noise tends to generate long sequence of low EAR values. It is necessary to filter out this type of noise, but EAR patterns of fast blinking should not be filtered out because people tend to fight eye fatigue by rapid series of blinking. Therefore, both the duration of consecutive low EAR values and the minimum EAR value in the duration need to be monitored. In an embodiment of the invention, EAR values are monitored and compared against a first threshold. If an EAR value immediately after a sequence of EAR values that are all greater than a first threshold drop below or is equal to the first threshold, then there will be some number of consecutive EAR values that are all equal to or less than the first threshold (FIG. 4). The time duration represented by the number of such consecutive EAR values is compared against a second threshold and a third threshold with the second threshold less than the third threshold. A spontaneous blink is detected when two conditions are satisfied. One condition is that the time duration is not less than the second threshold and not greater than the third threshold. The other condition is that at least one EAR value within the consecutive EAR values is equal to or less than a fourth threshold.

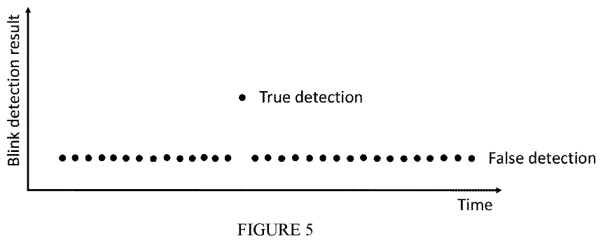

A detection result is generated for each EAR value to signalize either true or false blink detection. In an embodiment of the invention, in case a spontaneous blink is detected from a group of consecutive EAR values that satisfy the two detection conditions, a true blink detection result is generated for the last of the consecutive EAR values, and false blink detection result is generated for all other EAR values within the group. An example diagram of detection result is provided in FIG. 5 based on the EAR series in FIG. 4. For each person in the input images that are accepted, there will be a time series of detection result at the outputs of the blink detection function 130.

To support blink detection from images generated by multiple cameras, a plurality of the combination, which comprises a video camera, a facial recognition function, and a blink detection function as shown as 170 in FIG. 1, may be used to generate outputs to the data processing function 140.

The data processing function 140 calculates the rate of spontaneous blink from the input time series of blink detection results. An example of the blink rate (BR) is the number of spontaneous blink per minute. In that case, if a person reads or uses visual display device for 30 minutes, there will be 30 BR values. When the person is not reading or watching, the BR value will be zero. To store BR values efficiently, long time zeros can be replaced by the value of time duration. So in the data processing block 140, a person's BR record consists of multiple sequences of non-zero BR values separated by the values of time duration of no reading or watching activity. The record is transmitted to the statistic analysis function block 150.

Non-zero BR values are monitored and compared against a fifth threshold for each person. The data processing function 140 will send an interruption request to the eye stress relieving function 160 when a BR for that person exceeds the fifth threshold. The fifth threshold depends on the BR record of the person and the feedback from the statistic analysis function 150. It may be different for different people. Even for one person, it is dynamically adjusted so may change over time. The data processing function 140 may also receive the series of accepted face images of one or more persons from the blink detection function 130 and transmit them to the statistic analysis function 150 for detailed image analysis.

The statistic analysis function 150 contains a database for all participants. For each participant, the database include but not limited to age, gender, BR record, visual acuity test results, and information about general reading or watching environment. Large number of participants is preferred. Statistic analysis can be carried out to find the correlation between BR pattern in the BR records and progression of myopia indicated by visual acuity test results. The correlation will reveal the effectiveness of eye stress relieving that relies on the accuracy of the fifth threshold for each participant. The fifth threshold will depend on the statistic analysis results and the BR pattern. Using the received statistic analysis results from the statistic analysis function 150 and locally available BR record, an accurate fifth threshold can be calculated inside the data process function 140.

After receiving an interruption request from the data processing function 140 for one user, the eye stress relieving function 160 will send signals to the electronic display device which the camera is attached to or with built-in camera to generate visual messages to reminder that user of eye rest. Audio signal is also generated for the display device with built-in audio device. The necessity for the user to take break when seeing or hearing the reminder messages is justified by the accurate fifth threshold that is justified by the accurate correlation between BR pattern and myopia progression. Taking more frequent breaks than those suggested by the reminder messages is unnecessary for myopia prevention but taking less frequent breaks than those suggested by the reminder messages will lead to myopia development. In this manner, the myopia prevention method of the invention is effective and inexpensive.

Although the present invention has been described with reference to the disclosed embodiments, persons skilled in the art will recognize that changes may be made in form and detail without departing from the invention. Such modifications are well within the skill of those ordinarily skilled in the art. Accordingly, the invention is not limited except as by the appended claims.

We claim:

1. A system, comprising:
a facial recognition function;
a blink detection function;
a data processing function;
a statistic analysis function; and
an eye stress relieving function.

2. The system of claim 1 wherein the facial recognition function has input receiving stream of facial images from a video camera.

3. The video camera of claim 2 captures at least 10 frames per second, preferably 30 or more frames per second.

4. The facial recognition function of claim 2 detects all faces in the input image, extracts the faces, scales each face to a fixed size image and outputs the scaled images.

5. The system of claim 1 where the blink detection function has input receiving stream of images from the facial recognition function.

6. The blink detection function of claim 1 finds the location of facial landmarks in each input image, detects similarity of faces in the input images, rejects or accepts face images of a person, calculates the eye aspect ratio from landmarks around the eyes for accepted images, applies filter to improve the accuracy of spontaneous blink detection, and outputs blink detection results.

7. The values of the eye aspect ratio of claim 6 are arranged to a time series for all the accepted images of one person under the condition that the time order of the values match the time order of the images.

8. The extracted faces of claim 4 encompasses all facial landmarks of claim 6.

9. The filter of claim 6 compares the eye aspect ratio against a first threshold and counts the number of consecutive eye aspect ratios that are smaller or equal to the first threshold, compares the time duration represented by such sequence against a second threshold and a third threshold, and compares the eye aspect ratios within the sequence against a fourth threshold.

10. The number of consecutive eye aspect ratio of claim 9 is a local maximum in the sense that if there exists an eye aspect ratio immediately before or after the sequence, then that ratio is greater than the first threshold.

11. The second threshold of claim 9 is smaller than the third threshold of the same claim.

12. The block detection function of claim 6 outputs either true or false blink detection result for each eye aspect ratio.

13. The true blink detection result of claim 12 is generated if two conditions are satisfied, one of which is that the duration of the consecutive eye aspect ratio is not less than the second threshold and not greater than the third threshold of the same claim, and the other is that at least one of the consecutive eye aspect ratio is not greater than a fourth threshold.

14. The true blink detection result of claim 13 is generated for only one of the consecutive eye aspect ratio.

15. The blink detection function of claim 1 outputs a time series of blink detection result for each person whose face images are accepted to the data processing function of the same claim.

16. The data processing function of claim 1 calculates the frequency of spontaneous blink, which is the number of spontaneous blink in a fixed interval, from the time series of blink detection result at some of its inputs.

17. The data processing function of claim 16 outputs blink rate information, which consists of multiple sequences of non-zero blink rate values separated by the values of time duration of no reading or watching activity, to the statistic analysis function of claim 1.

18. The data processing function of claim 16 compares the frequency of spontaneous blink to a fifth threshold for a person and outputs an interruption request to the eye stress relieving function of claim 1.

19. The data processing function of claim 16 conditionally receives the series of accepted face images of one or more persons from the blink detection function of claim 6 and transmit them to the statistic analysis function of claim 1 for detailed image analysis.

20. A plurality of the combination, which comprises a video camera, a facial recognition function, and a blink detection function of claim 1, may be used to generate outputs to the data processing function of claim 1.

21. The statistic analysis function of claim 17 contains a database for all participants, which includes but not limited to age, gender, blink rate information, visual acuity test results, and information about general reading or watching environment for each participant.

22. The statistic analysis function of claim 21 performs statistic analysis, generates correlation between blink rate and progression of myopia indicated by visual acuity test results, calculates parameters to generate an accurate fifth threshold for each participant, and outputs the calculated parameters to the data processing function of claim 18 for the calculation of the fifth threshold.

23. The eye stress relieving function of claim 18 sends reminder messages to be displayed on a visual display terminal after it receives interruption request from the data processing function of the same claim.

24. The reminder messages of claim 23 may include audio signals to be played on a visual display terminal.
